(19) United States
(12) Patent Application Publication
(10) Pub. No.: US 2022/0002260 A1
(43) Pub. Date: Jan. 6, 2022

(54) PREPARATION OF 2-SUBSTITUTED 4-METHYL-TETRAHYDROPYRANS FROM 2-SUBSTITUTED 4-HYDROXY-4-METHYL-TETRAHYDROPYRANS AS STARTING MATERIALS

(71) Applicant: BASF SE, Ludwigshafen am Rhein (DE)

(72) Inventors: Christoph STOCK, Ludwigshafen am Rhein (DE); Irene DE WISPELAERE, Antwerpen (BE); Bernhard BRUNNER, Ludwigshafen am Rhein (DE); Wolfgang KRAUSE, Lampertheim (DE); Florian GARLICHS, Lampertheim (DE)

(21) Appl. No.: 17/290,160

(22) PCT Filed: Oct. 28, 2019
(86) PCT No.: PCT/EP2019/079322

§ 371 Date: Apr. 29, 2021

(51) Int. Cl. C07D 309/04; B01J 23/44; B01J 21/18

(52) U.S. Cl. CPC C07D 309/04; B01J 21/18; B01J 23/44

(57) ABSTRACT

The present invention relates to a method for preparing 2-substituted 4-methyltetrahydropyrans from 2-substituted 4-hydroxy-4-methyltetrahydropyrans as starting materials.

### BACKGROUND OF THE INVENTION

The present invention relates to a method for preparing 2-substituted 4-methyltetrahy-dropyrans from 2-substituted 4-hydroxy-4-methyltetrahydropyrans as starting materials.

### PRIOR ART

Alkyl-substituted tetrahydropyrans have found widespread use as aromas and flavorings. A well-known representative of this class is 2-isobutyl-4-methyltetrahydropyran (dihydrorose oxide or Dihydrorosan®), which has a floral, green scent note.

The first synthesis of dihydrorose oxide was described by M. Julia and B. Jacquet, Bulletin de la Societe Chimique de France 1963, 8-9, 1983. Starting from but-2-en-1-al, a cyclic acetal was obtained by a Diels-Alder reaction with ethyl vinyl ether and subsequent hydrogenation. After elimination of ethanol, hydrobromination of the resulting double bond and a final Grignard reaction with isopropylmagnesium bromide, a racemic mixture of cis- and trans-dihydrorose oxide was obtained.

Liu et al. in J. Heterocyclic Chem, 21, 129-132 (1984) describe the preparation of cis-dihy-drorose oxide by hydrogenation of 2-isobutyl-4-methyl-5,6-dihydro-4H-pyran with PtO₂ in acetic acid.

Schindler and Vogel describe schematically in Perfume & Flavorist, vol. 11, 29-30 (1986) the preparation of dihydrorose oxide from 3-methylbut-3-en-1-ol and 3-methylbutanal as starting material, in which a cis/trans mixture is obtained in a ratio of 70:30. Neither the reaction route nor the conditions to be observed are described in more detail.

EP 0 770 670 B1 describes a perfume composition comprising 2-substituted (4R)-cis-4-methyltetrahydro-2H-pyrans. In the application, the odor properties of the isomers of rose oxide and dihydrorose oxide are described. The isomers of dihydrorose oxide are synthesized by hydrogenation of the corresponding isomers of rose oxide.

WO 2014/060345 describes a method for the preparation of 2-substituted 4-hydroxy-4-methyltetrahydropyrans and 2-substituted 4-methyltetrahydropyrans by reacting iso-prenol (3-methylbut-3-enol) with an aldehyde. In the first step, isoprenol is reacted in the presence of a suitable aldehyde, wherein a mixture of 2-substituted 4-hydroxy-4-methyltetrahydropyrans, 6-substituted 4-methyl-3,6-dihydro-2H-pyrans, 2-substituted 4-methylenetetrahydropyrans, 2-substituted 4-methyl-3,6-dihydro-2H-pyrans and 2-substituted 4,4-dimethyl-1,3-dioxanes is obtained. The alcohol compound is separated off. The remaining compounds are subjected to hydrogenation to give 2-substituted 4-methyltetrahydropyrans and dioxane compounds.

WO 2015/158584 describes a method for the preparation of 2-substituted 4-hydroxy-4-methyltetrahydropyrans. 2-substituted 4,4-dimethyl-1,3-dioxanes are reacted with a strong acid, wherein a product mixture of 6-substituted 4-methyl-3,6-dihydro-2H-pyrans, 2-substituted 4-methylenetetrahydropyrans and 2-substituted 4-methyl-3,6-dihydro-2H-pyrans is obtained. The product mixture is subjected to hydrogenation.

Both US 2009/0263336 and EP 2 112 144 describe the preparation of 2-alkyl-4-methyl-tetrahydropyranol compounds. The pyranol obtained can be converted by dehydration in a further step to a mixture of 4-methylene-2-alkyltetrahydropyran, 4-methyl-2-alkyl-5,6-dihydropyran and 4-methyl-2-alkyl-3,6-dihydropyran. The mixture obtained can optionally be hydrogenated to afford the corresponding 4-methyl-2-alkyltetrahydropyrans. Firstly, the methods in these documents were not a one-pot synthesis. Each intermediate compound must be isolated for the next step. Secondly, the 2-alkyl-4-methyltetrahydropyran derivatives are prepared in the documents mentioned from a mixture of the aforementioned compounds by hydrogenation. There is no mention of an acid catalyst. The acid mentioned in these documents is used only in the preparation of the pyranol.

There continues to be a great need for effective methods for preparing 2-substituted 4-methyltetrahydropyrans from readily available starting materials. In addition to the synthesis from pure substances, the use of previously unusable by-products from other synthetic methods is of particular interest in this case.

It is an object of the present invention to provide an improved method for preparing 2-substituted 4-methyltetrahydropyrans.

Surprisingly, it has now been found that, by hydrogenating 2-substituted 4-hydroxy-4-methyltetrahydropyrans in the presence of a hydrogenation catalyst under acidic conditions, these compounds can be converted by a quick route to 2-substituted 4-methyltetrahydropyrans, specifically dihydrorose oxide.

### SUMMARY OF THE INVENTION

The invention relates to a method for preparing compounds of the general formula (I)

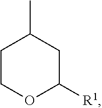

where R¹ is selected from p1 straight-chain or branched C₁-C₁₂-alkyl, where alkyl is unsubstituted or has at least one substituent selected from aryl, C₁-C₁₂-alkoxy and C₁-C₁₂-alkylcarbonyl,
cycloalkyl having a total of 3 to 20 carbon atoms that is unsubstituted or substituted by 1, 2, 3 or 4 substituents selected from C₁-C₁₂-alkyl, C₁-C₁₂-alkoxy, C₁-C₁₂-alkyl, C₁-C₁₂-alkoxy, phenyl and benzyl, comprising the steps of:

a) providing at least one compound of the general formula (II)

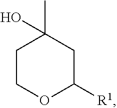

where R¹ is as defined above,

b) hydrogenating the compound (II) in the presence of a hydrogenation catalyst under acidic conditions,

wherein it is a one-pot synthesis.

The invention further relates to a method for preparing compounds of the general formula (I)

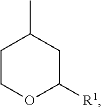

where R¹ is selected from straight-chain or branched C₁-C₁₂-alkyl, where alkyl is unsubstituted or has at least one substituent selected from aryl, C₁-C₁₂-alkoxy and C₁-C₁₂-alkylcarbonyl,
cycloalkyl having a total of 3 to 20 carbon atoms that is unsubstituted or substituted by 1, 2, 3 or 4 substituents selected from C₁-C₁₂-alkyl, C₁-C₁₂-alkoxy, C₁-C₁₂-alkyl, C₁-C₁₂-alkoxy, phenyl and benzyl, comprising the steps of:

a) providing at least one compound of the general formula (II)

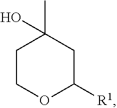

where R¹ is as defined above,

b) hydrogenating the compound (II) in the presence of a hydrogenation catalyst under acidic conditions.

### DESCRIPTION OF THE INVENTION

The method according to the invention has the following advantages:
The reaction provided according to the invention allows access to 2-substituted 4-methyltetrahydropyrans and specifically to dihydrorose oxide/Dihydrorosan®, which requires only one reaction step (one-pot synthesis).
The preparation of the 2-substituted 4-methyltetrahydropyrans, specifically dihydrorose oxide, avoids the use of further expensive and/or potentially hazardous reagents such as Grignard reagents or complex hydrides such as lithium aluminum hydride.

In the context of the invention, one-pot synthesis describes a synthesis which requires only one reaction step. There is no isolation of intermediates. The reaction according to the invention takes place in situ. In other words, all of the substances required for the method according to the invention in steps a) and b) are already present in the reaction vessel from the start or are added in the course of the reaction, but without stopping the reaction. As soon as the reaction is complete, the desired product is obtained. The product can optionally be purified by the customary purification methods known to those skilled in the art, such as filtration, distillation, extraction or a combination thereof.

Unless otherwise specified in more detail below, the terms “2-substituted 4-methyltetrahydropyran”, “2-(2-methylpropyl)-4-methyltetrahydropyran ran”, “2-isobutyl-4-methyltetrahydropyran” (=“dihydrorose oxide” or “Dihydrorosan®”), “2-substituted 4-hydroxy-4-methyltetrahydropyran”, “2-(2-methylpropyl)-4-hydroxy-4-methyltetrahydropyran” and “2-isobutyl-4-methyltetrahydropyran-4-ol” refer in the context of the invention to cis/trans mixtures of any composition and also to the pure conformational isomers. The terms mentioned above additionally refer to all enantiomers in pure form and to racemic and optically active mixtures of the enantiomers of these compounds.

Where cis and trans diastereoisomers of the compounds (I) or (II) are being discussed hereinafter, only one of the enantiomeric forms is shown in each case. The isomers of 2-(2-methylpropyl)-4-methyltetrahydropyran (I) (dihydrorose oxide/Dihydrorosan®) are shown below for illustration purposes only:

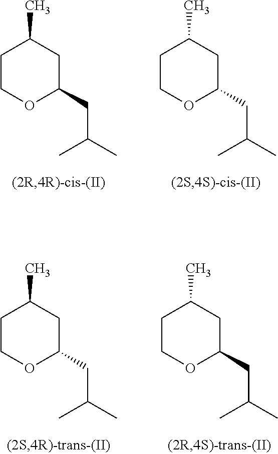

In the context of the present invention, the expression straight-chain or branched alkyl preferably means C₁-C₆-alkyl and particularly preferably C₁-C₄-alkyl. In particular, alkyl is methyl, ethyl, n-propyl, isopropyl, n-butyl, isobutyl (2-methylpropyl), sec-butyl (1-methylpropyl), tert-butyl (1,1-dimethylethyl), n-pentyl or n-hexyl. Alkyl is especially methyl, ethyl, n-propyl, isopropyl or isobutyl.

In the context of the present invention, the expression alkyl substituted by aryl preferably means C₁-C₆-alkyl substituted by aryl and particularly preferably C₁-C₄-alkyl substituted by aryl. Alkyl substituted by aryl is in particular benzyl, 1-phenethyl or 2-phenethyl.

In the context of the present invention, the expression straight-chain or branched alkoxy preferably means C₁-C₆-alkoxy and particularly preferably C₁-C₄-alkoxy. In particular, alkoxy is methoxy, ethoxy, n-propyloxy, isopropyloxy, n-butyloxy, isobutyloxy, sec-butyloxy, tert-butyloxy, n-pentyloxy or n-hexyloxy. Alkoxy is especially methoxy, ethoxy, n-propyloxy, isopropyloxy, or isobutyloxy.

In the context of the invention, cycloalkyl refers to a cycloaliphatic radical preferably having 3 to 10, particularly preferably 5 to 8, carbon atoms. Examples of cycloalkyl groups are particularly cyclopropyl, cyclobutyl, cyclopentyl, cyclohexyl, cycloheptyl or cyclooctyl. Cycloalkyl is especially cyclohexyl.

Substituted cycloalkyl groups may have one or more substituents (e.g. 1, 2, 3, 4 or 5), depending on the size of the ring. These are preferably each independently selected from C₁-C₆-alkyl, C₁-C₆-alkoxy, phenyl and benzyl, particularly preferably C₁-C₆-alkyl and C₁-C₆-alkoxy. In the case of substitution, the cycloalkyl groups preferably bear one or more, for example one, two, three, four or five, C₁-C₆-alkyl groups. Examples of substituted cycloalkyl groups are particularly 2- and 3-methylcyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl and 2-, 3- and 4-isobutylcyclohexyl.

In the context of the invention, the expression alkylcarbonyl preferably means (C₁-C₆-alkyl)carbonyl, where alkyl, as defined above, is bonded to the rest of the molecule via a carbonyl group.

In the context of the present invention, the expression “aryl” encompasses mono- or polycyclic aromatic hydrocarbon radicals typically having 6 to 18, preferably 6 to 14, particularly preferably 6 to 10, carbon atoms. Examples of aryl are particularly phenyl, naphthyl, indenyl, fluorenyl, anthracenyl, phenanthrenyl, naphthacenyl, chrysenyl, pyrenyl, etc., and especially phenyl or naphthyl.

Substituted aryls may have one or more substituents (e.g. 1, 2, 3, 4 or 5), depending on the number and size of their ring systems. These are each preferably independently selected from C₁-C₆-alkyl and C₁-C₆-alkoxy. Examples of substituted aryl radicals are 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triiso-propylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl, 2,4,6-tri-tert-butylphenyl, 1-methyl-2-naphthyl, 3-methyl-2-naphthyl, 1,3-dimethyl-2-naphthyl, 5,6,7,8-tetramethyl-2-naphthyl, 5-methyl-2-naphthyl, 6-methyl-2-naphthyl, 7-methyl-2-naphthyl, 8-methyl-2-naphthyl.

R¹ in the compounds of the formula (I) and (II) is preferably straight-chain or branched C₁-C₁₂-alkyl, where alkyl is unsubstituted or substituted by aryl. R¹ is particularly preferably straight-chain or branched C₁-C₆-alkyl, where alkyl is unsubstituted or has at least one substituent selected from phenyl and C₁-C₆-alkoxy.

Preferred definitions in accordance with the invention for the radical R¹ therefore are, for example, methyl, ethyl, n-propyl, isopropyl, n-butyl, isobutyl, n-pentyl, n-hexyl or n-heptyl, preferably methyl, ethyl, n-propyl, isopropyl, n-butyl, isobutyl, especially preferably isobutyl (2-methylpropyl).

The present invention therefore relates in the context of a preferred embodiment to a method for the preparation and isolation of 2-(2-methylpropyl)-4-methyltetrahydropyran of the formula (Ia) (dihydrorose oxide/Dihydrorosan®).

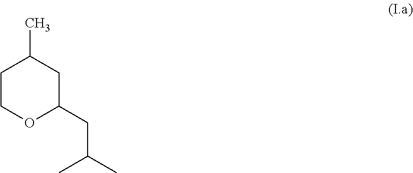

### Step a)

Suitable starting materials for use in step a) may be at least one compound of the formula (II)

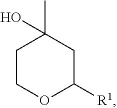

where R¹ is selected from straight-chain or branched C₁-C₁₂-alkyl, where alkyl is unsubstituted or has at least one substituent selected from aryl, C₁-C₁₂-alkoxy and C₁-C₁₂-alkylcarbonyl, cycloalkyl having a total of 3 to 20 carbon atoms that is unsubstituted or substituted by 1, 2, 3 or 4 substituents selected from C₁-C₁₂-alkyl, C₁-C₁₂-alkoxy,C₁-C₁₂-alkyl, C₁-C₁₂-alkoxy, phenyl and benzyl.

R¹ is preferably selected from a straight-chain or branched C₁-C₆-alkyl, where alkyl is unsubstituted or has at least one substituent selected from phenyl and C₁-C₆-alkoxy.

R¹ is particularly preferably selected from methyl, ethyl, n-propyl, isopropyl, n-butyl, iso-butyl, n-pentyl, n-hexyl and phenyl.

R¹ is especially preferably selected from methyl, ethyl, n-propyl, isopropyl, n-butyl, iso-butyl, n-pentyl and n-hexyl.

In a specific embodiment, R¹ is isobutyl (2-methylpropyl).

The synthetic route for the preparation of the compound of the formula (II) is described in WO 2010/133473, WO 2015/158454 and WO 2014/060345.

Step b)

In accordance with the invention, the compound of the formula (II) is subjected to an elimination followed by hydrogenation in the presence of a hydrogenation catalyst under acidic conditions. The elimination and hydrogenation in step b) converts the compound of the formula (II) to the corresponding compound of the formula (I).

The elimination followed by hydrogenation is preferably carried out in one reaction stage (one-pot synthesis), i.e. without isolation of intermediate compounds.

In the context of the invention, the expression “under acidic conditions” is understood to mean that the reaction takes place in the presence of an acid. Acid is understood to mean any substance which has Bronsted or Lewis acidity.

Such substances are preferably selected from proton donors, electron acceptors and mixtures thereof.

Proton donors are preferably selected from molecular protic acids, ion exchangers and mixtures thereof.

Electron acceptors are preferably selected from acidic molecular element compounds, oxidic acidic solids and mixtures thereof.

Suitable molecular protic acids are, for example, hydrochloric acid, sulfuric acid, phosphoric acid, nitric acid, acetic acid, formic acid, trifluoromethanesulfonic acid, methanesulfonic acid, p-toluenesulfonic acid and mixtures thereof.

Suitable acidic molecular element compounds are, for example, aluminum chloride, boron trifluoride, zinc chloride, phosphorus pentafluoride, arsenic trifluoride, tin tetrachloride, titanium tetrachloride, antimony pentafluoride and mixtures thereof.

Suitable oxidic acidic solids are, for example, zeolites, silicates, aluminates, aluminosilicates, clays and mixtures thereof.

Suitable ion exchangers are acidic cationic ion exchangers.

In the context of the present invention, the expression “acidic cation exchanger” is understood to mean those cation exchangers in the H⁺ form that have acidic groups, usually sulfonic acid groups, the matrix of which can be gel-like or macroporous. A preferred embodiment of the method according to the invention is accordingly characterized in that an acidic cation exchanger containing or comprising sulfonic acid groups is used.

Acidic cation exchangers are, in particular, ion-exchange resins in the H⁺ form. Useful examples of these include:
acidic ion exchangers (such as Amberlyst, Amberlite, Dowex, Lewatit, Purolite, Serdolit), which are based on polystyrene and which comprise copolymers of styrene and divinylbenzene as a support matrix having sulfonic acid groups in H⁺ form,
ion exchange groups functionalized with sulfonic acid groups (—SO₃H).

The ion exchangers differ in the structure of their polymer skeletons and a distinction is made between gel-like and macroporous resins. The acidic ion-exchange resins are generally regenerated using hydrochloric acid and/or sulfuric acid.

Nafion® is the Dupont company name for perfluorinated polymeric ion-exchange resins. These are perfluorinated ion-exchange materials consisting of fluorocarbon-based chains and perfluorinated side chains comprising sulfonic acid groups. The resins are produced by copolymerization of perfluorinated, terminally unsaturated and sulfonyl fluoride-functionalized ethoxylates with perfluoroethene. Nafion® is one of the gel-like ion-exchange resins. An example of one such perfluorinated polymeric ion-exchange resin is Nafion®>NR-50.

The acidic cation exchangers are generally used in the H⁺ form, the ion exchanger comprising a polymer skeleton containing sulfonic acid groups and being either in gel form or comprising macroporous resins.

A very particularly preferred embodiment of the method according to the invention is characterized in that the ion exchanger is based on a polystyrene skeleton having sulfonic acid groups or on a perfluorinated ion-exchange resin having sulfonic acid groups.

The commercially available acidic cation exchangers are known under the trade names Lewatit® (Lanxess), Purolite® (The Purolite Company), Dowex® (Dow Chemical Company), Amberlite® (Rohm and Haas Company), Amberlyst™ (Rohm and Haas Company). Acidic cation exchangers preferred according to the invention include, for example: Lewatit® K 1221, Lewatit® K 1461, Lewatit® K 2431, Lewatit® K 2620, Lewatit® K 2621, Lewatit® K 2629, Lewatit® K 2649, Amberlite® IR 120, Amberlyst™ 131, Amberlyst™ 15, Amberlyst™ 31, Amberlyst™ 35, Amberlyst™ 36, Amberlyst™ 39, Amberlyst™ 46, Amberlyst™ 70, Purolite® SGC650, Purolite® C100H, Purolite® C150H, Dowex® 50X8, Dowex® 88, Serdolit® red and Nafion® NR-50.

In the context of a preferred embodiment, the reaction of compound (II) to be carried out in accordance with the invention is carried out in the presence of at least one acidic cation exchanger selected from the group of cation exchangers comprising Lewatit® K 1221, Lewatit® K 2629, Amberlyst™ 131, Amberlyst™ 35, Purolite® SGC650, Purolite® C100H, Purolite® C15OH, Amberlite® IR 120, Dowex® 88 and Dowex® 50X8.

Acidic cation exchangers particularly preferred according to the invention are the cation exchangers Amberlyst™ 35, Dowex® 88 and/or Amberlite® IR 120.

An acidic cation exchanger very particularly preferred according to the invention is Amberlyst™ 35 which, like the other cation exchangers mentioned, is commercially available.

The acidic ion-exchange resins are generally regenerated using hydrochloric acid and/or sulfuric acid.

The hydrogenation in step b) may be carried out in a conventional manner using a hydrogenation catalyst of the prior art. The hydrogenation may be carried out catalytically either in the gas phase or in the liquid phase. The hydrogenation in step b) is preferably carried out in the liquid phase in the presence of a heterogeneous hydrogenation catalyst and a hydrogen-containing gas.

Suitable hydrogenation catalysts include, in principle, all homogeneous and heterogeneous catalysts suitable for hydrogenating unsaturated organic compounds. These include, for example, metals, metal oxides, various metal compounds thereof or mixtures thereof. Suitable hydrogenation catalysts preferably comprise at least one transition metal, preferably from the transition groups I and VI to VIII of the periodic table of the elements. These preferably include Pd, Pt, Ni, Rh, Ru, Co, Fe, Zn, Cu, Re or mixtures thereof.

The hydrogenation catalyst may comprise at least one further metal/element which is different from the metals described above. The further metal/element is preferably selected from alkali metals, alkaline earth metals, aluminum, silicon, lanthanoids and mixtures thereof.

The proportion of the further metal/element is preferably in the range from 0.1% to 10% by weight based on the total weight of the active part of the hydrogenation catalyst (excluding the support).

The catalysts may consist solely of the active components, or the active components may be applied to supports. Suitable support materials are, e.g. zirconium dioxide, barium oxide, zinc oxide, magnesium oxide, titanium oxide, aluminum oxide, TiO₂—Al₂O₃, ZrO₂-Al₂O₃, zeolites, hydrotalcite, silicon carbide, tungsten carbide, silicon dioxide, carbon, especially activated carbon or sulfated carbon, diatomaceous earth, clay, barium sulfate, calcium carbonate and mixtures.

In one embodiment, the support materials at the same time comprise an acid used according to the invention or consist thereof.

To increase the catalytic activity, Ni, Cu or Co, including in the form of Raney catalysts, Pd, Pt, Rh, Ru, Co, Fe, Zn, Cu, or mixtures thereof, can be used in the form of a metal sponge having a very large surface area.

Palladium on carbon, palladium on Al₂O₃, palladium on SiO₂ or platinum on carbon is preferably used advantageously for the hydrogenation in step b) of the method according to the invention. Palladium on carbon is particularly preferably used advantageously.

Other suitable catalysts comprise, for example, 80% to 100% by weight of nickel and/or cobalt and up to 20% by weight of activating metals such as copper and/or chromium. Such catalysts are particularly advantageously used as supported catalysts.

The content of catalytically active metals in such supported catalysts where the support material is carbon is generally from 0.05% to 10% by weight based on the sum of the catalytically active metals and support.

The content of catalytically active metals in such supported catalysts where the support material is an oxide, e.g. Al₂O₃ or SiO₂, is generally 0.01 to 1% by weight based on the sum of the catalytically active metals and support.

The catalysts for the hydrogenation in step b) may be used in the form of shaped bodies. Examples comprise catalyst extrudates such as ribbed extrudates and other extrudate forms, eggshell catalysts, tablets, rings, spheres, spall, etc.

Preference is given to carrying out the hydrogenation in step b) at a temperature of 60 to 200° C., preferably 120 to 150° C., especially 135 to 145° C.

When the reaction is carried out in the gas phase, the pressure is preferably within a range from 0.9 to 50 bar, particularly preferably 1 to 20 bar.

When the reaction is carried out in the liquid phase, the pressure is preferably within a range from 0.9 to 200 bar, particularly from 40 to 80 bar.

The hydrogenation in step b) can be carried out in one reactor or in a plurality of reactors connected in series. The hydrogenation can be effected continuously or batchwise. For batchwise hydrogenation, a pressure vessel for example may be used. Suitable pressure vessels are, for example, autoclaves equipped with an apparatus for heating and for stirring the reactor contents. The hydrogenation is preferably carried out in the liquid phase over a fixed bed, preferably in liquid-phase mode or trickle mode, or in the form of a suspension catalysis. Operation in fixed-bed mode can be conducted, for example, in liquid-phase mode or in trickle mode. In this case, the catalysts are preferably used in the form of shaped bodies, for example in the form of pressed cylinders, tablets, pellets, wagonwheels, rings, stars, or extrudates such as solid extrudates, polylobal extrudates, hollow extrudates, honeycombs etc.

In suspension mode, heterogeneous catalysts are likewise used. The heterogeneous catalysts are usually used in a finely divided state and are in fine suspension in the reaction medium.

In the case of hydrogenation over a fixed bed, a reactor with a fixed bed arranged in the interior thereof, through which the reaction medium flows, is used. The fixed bed may be formed from a single bed or from a plurality of beds. Each bed may have one or more zones, at least one of the zones comprising a material active as a hydrogenation catalyst. Each zone may have one or more different catalytically active materials and/or one or more different inert materials. Different zones may each have identical or different compositions. It is also possible to provide a plurality of catalytically active zones separated from one another, for example, by inert beds. The individual zones may also have different catalytic activity. To this end, it is possible to use different catalytically active materials and/or to add an inert material to at least one of the zones. The reaction medium which flows through the fixed bed, according to the invention, comprises at least one liquid phase. The reaction medium may also additionally comprise a gaseous phase.

Reactors used for the hydrogenation in suspension are especially loop apparatuses such as jet loops or propeller loops, stirred-tank reactors, which may also be configured as stirred-tank reactor cascades, bubble columns or airlift reactors.

The hydrogenation in step b) is preferably carried out in suspension mode.

The hydrogenation can be carried out with or without addition of a solvent. Useful solvents include alcohols, ethers and hydrocarbons, for example methanol, ethanol, isopropanol, dioxane, tetrahydrofuran, n-pentane, hexane, cyclohexane, toluene, etc. The hydrogenation in step b) is preferably carried out without addition of a solvent.

For the hydrogenation in step b), the compound of the formula (II) obtained in step a) can be contacted with a hydrogen-containing gas and a hydrogenation catalyst. Suitable hydrogen-containing gases are selected from hydrogen and mixtures of hydrogen with at least one inert gas. Suitable inert gases are, for example, nitrogen or argon. For the hydrogenation in step b), hydrogen is preferably used in undiluted form, typically at a purity of about 99.9% by volume.

The hydrogenation in step b) converts the compounds of the formula (II) to 2-substituted 4-methyltetrahydropyrans (I). The starting material used for the hydrogenation preferably comprises compounds of the formula (II), where the radical R¹ is as defined above. R¹ is preferably isobutyl.

In a specific embodiment, the hydrogenation in step b) converts the compounds (II) to 2-isobutyl-4-methyl-tetrahydropyran (Ia) (dihydrorose oxide).

The compound of the formula (I) obtained in step b) preferably has a diastereomeric ratio of the cis-diastereomer to the trans-diastereomer within a range from 10:90 to 90:10, particularly preferably from 65:35 to 90:10.

The compound of the formula (I) obtained in step b) can be converted to a form suitable for commercial use by simple purification steps.

If desired, the compound of the formula (I) obtained in step b) can be subjected to further processing. For this purpose, the compound (I) obtained in step b) can in principle be subjected to customary purification processes known to those skilled in the art. This includes, for example, filtration, neutralization, distillation, extraction or a combination thereof.

A fraction enriched in 2-substituted 4-methyltetrahydropyrans (I) and a fraction depleted in 2-substituted 4-methyltetrahydropyrans (I) are preferably isolated from the hydrogenation product obtained in step b).

The compound (I) obtained in step b) is preferably subjected to a distillative separation. Suitable apparatuses for distillative separation comprise distillation columns such as tray columns, which may be equipped with bubble-caps, sieve plates, sieve trays, structured packings, random packings, valves, side draws, etc., evaporators such as thin-film evaporators, falling-film evaporators, forced-circulation evaporators, Sambay evaporators etc. and combinations thereof.

The compound (I) obtained in step b) is preferably subjected in step c) to a distillative separation in at least one distillation column which is provided with separating internals.

A fraction enriched in 2-substituted 4-methyl-tetrahydropyrans (I) is preferably isolated in step c) from the compound (I) obtained in step b), the diastereomeric ratio of the cis-diastereomer to the trans-diastereomer being within a range of 10:90 to 90:10, preferably of 65:35 to 90:10.

To remove further water-soluble impurities, the fraction enriched in 2-substituted 4-methyltetrahydropyrans (I) obtained in step c) may be subjected to at least one wash step with water. Alternatively or in addition, the fraction enriched in 2-substituted 4-methyltetrahydropyrans (I) obtained in step c) may be subjected to a further distillative purification.

The examples that follow serve to elucidate the invention without restricting it in any way.

### EXAMPLES Gas chromatographic analyses were carried out in accordance with the following method:

Column: DB WAX 30 m×0.32 mm;

FT 0.25 μm;

Injector temperature: 200° C.; detector temperature 280° C.;

Temperature program: Starting temp.: 50° C., at 3° C./min to 170° C., at 20° C./min to 230° C., 7 min isothermal;

Retention times: 2-lsobutyl-4-methyltetrahydropyran-4-ol t_{R}=28.9 min and 30.4 cis-Dihydrorose oxide t_{R}=8.77 min trans-Dihydrorose oxide t_{R}=10.09 min

Concentrations of the resulting crude products (% by weight) were determined by GC analysis using an internal standard.
1. Preparation of 2-isobutyl-4-methyltetrahydropyran starting from 2-isobutyl-4-methyltetrahydropyran-4-ol in methanol

12 g of 2-isobutyl-4-methyltetrahydropyran-4-ol (isomeric ratio 24:76), 28 g of methanol, 0.2 g of Pd/C catalyst (10% Pd on C) and 0.2 g of Amberlyst 35 dry are weighed into an autoclave. This is closed and flushed once each with nitrogen and hydrogen. The autoclave is first pressurized with 30 bar of hydrogen, then heated to 140° C. and, after the reaction temperature has been reached, the pressure is adjusted to 50 bar. The experiment is stirred under these conditions for 12 hours, the autoclave being repressurized with hydrogen after 1 h, 3 h and 5 h in order to keep the pressure at 50 bar. The autoclave is then depressurized and cooled. The catalyst and ion exchanger are filtered off the resulting solution being colorless and clear.

At a 2-isobutyl-4-methyltetrahydropyran-4-ol conversion of >99%, 2-isobutyl-4-methyl-tetrahydropyran was formed with a selectivity of 86% with respect to 2-isobutyl-4-methyltetrahydropyran-4-ol. The cis/trans ratio is 5.33:1.
2. Preparation of 2-isobutyl-4-methyltetrahydropyran starting from solvent-free 2-isobutyl-4-methyltetrahydropyran-4-ol
   36 g of 2-isobutyl-4-methyltetrahydropyran-4-ol (isomeric ratio 24:76), 0.4 g of Pd/C catalyst (10% Pd on C) and 0.4 g of Amberlyst 35 dry are weighed into an autoclave. This is closed and flushed once each with nitrogen and hydrogen. The autoclave is first pressurized with 30 bar of hydrogen, then heated to 140° C. and, after the reaction temperature has been reached, the pressure is adjusted to 50 bar. The experiment is stirred under these conditions for 12 hours, the autoclave being repressurized with hydrogen after 1 h, 3 h and 5 h in order to keep the pressure at 50 bar. The autoclave is then depressurized and cooled. The catalyst and ion exchanger are filtered off, the resulting solution being colorless and clear.

At a 2-isobutyl-4-methyl-tetrahydropyran-4-ol conversion of 60%, 2-isobutyl-4-methyltetrahydropyran was formed with a selectivity of 50% with respect to 2-isobutyl-4-methyltetrahydropyran-4-ol. The cis/trans ratio is 6.1:1.
3. Preparation of 2-isobutyl-4-methyltetrahydropyran starting from 2-isobutyl-4-methyltetrahydropyran-4-ol in methanol

12 g of 2-isobutyl-4-methyltetrahydropyran-4-ol (isomeric ratio 24:76), 28 g of methanol, 0.4 g of Pd/C catalyst (5% Pd on C) and 0.4 g of Amberlyst 35 dry are weighed into an autoclave. This is closed and flushed once each with nitrogen and hydrogen. The autoclave is first pressurized with 30 bar of hydrogen, then heated to 140° C. and, after the reaction temperature has been reached, the pressure is adjusted to 50 bar. The experiment is stirred under these conditions for 12 hours, the autoclave being repressurized with hydrogen after 1 h, 3 h and 5 h in order to keep the pressure at 50 bar. The autoclave is then depressurized and cooled. The catalyst and ion exchanger are filtered off, the resulting solution being colorless and clear.

At a 2-isobutyl-4-methyltetrahydropyran-4-ol conversion of >81.2%, 2-isobutyl-4-methyltetrahydropyran was formed with a selectivity of 65.6% with respect to 2-isobutyl-4-methyltetrahydropyran-4-ol. The cis/trans ratio is 6.06:1.
4. Preparation of 2-isobutyl-4-methyltetrahydropyran starting from 2-isobutyl-4-methyltetrahydropyran-4-ol in methanol

12 g of 2-isobutyl-4-methyltetrahydropyran-4-ol (isomeric ratio 24:76), 28 g of methanol, 0.7 g of Pd/C catalyst (5% Pd on C) and 0.7 g of Amberlyst 35 dry are weighed into an autoclave. This is closed and flushed once each with nitrogen and hydrogen. The autoclave is first pressurized with 30 bar of hydrogen, then heated to 120° C. and, after the reaction temperature has been reached, the pressure is adjusted to 80 bar. The experiment is stirred under these conditions for 12 hours, the autoclave being repressurized with hydrogen after 1 h, 3 h and 5 h in order to keep the pressure at 50 bar. The autoclave is then depressurized and cooled. The catalyst and ion exchanger are filtered off, the resulting solution being colorless and clear.

At a 2-isobutyl-4-methyl-tetrahydropyran-4-ol conversion of 56.7%, 2-isobutyl-4-methyltetrahydropyran was formed with a selectivity of 62% with respect to 2-isobutyl-4-methyltetrahydropyran-4-ol. The cis/trans ratio is 5.17:1.

1-13. (canceled)

14. A method for preparing compounds of the general formula (I) 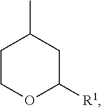
where R¹ is selected from
straight-chain or branched C₁-C₁₂-alkyl, where alkyl is unsubstituted or has at least one substituent selected from aryl, C₁-C12-alkoxy and C₁-C₁₂-alkylcarbonyl,
cycloalkyl having a total of 3 to 20 carbon atoms that is unsubstituted or substituted by 1, 2, 3 or 4 substituents selected from C₁-C₁₂-alkyl, C₁-C₁₂-alkoxy, C₁-C₁₂-alkyl, C₁-C₁₂-alkoxy, phenyl and benzyl,
comprising the steps of:
a) providing at least one compound of the general formula (II) 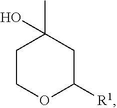
where R¹ is as defined above,
b) hydrogenating the compound (II) in the presence of a hydrogenation catalyst under acidic conditions,
wherein the method is a one-pot synthesis.

15. The method according to claim 14, where R¹ is a straight-chain or branched C₁-C₆-alkyl which is unsubstituted or has at least one substituent selected from phenyl and C₁-C₆-alkoxy.

16. The method according to claim 14, where R¹ is methyl, ethyl, n-propyl, isopropyl, n-butyl, isobutyl, n-pentyl, n-hexyl or phenyl.

17. The method according to claim 16, wherein R¹ is isobutyl.

18. The method according to claim 14, wherein the isomeric ratio of cis:trans of compound (I) is in the range from 10:90 to 90:10.

19. The method according to claim 18, wherein the isomeric ratio of cis:trans of compound (I) is in the range from 65:35 to 90:10.

20. The method according to claim 14, wherein the hydrogenation in step b) is carried out in the presence of an acid selected from at least one protic acid, at least one Lewis acid, at least one acidic ion exchanger, at least one oxidic acidic solid, at least one acidic molecular element compound and mixtures thereof.

21. The method according to claim 14, wherein the hydrogenation in step b) is carried out in the presence of an acid selected from the group consisting of hydrochloric acid, sulfuric acid, phosphoric acid, nitric acid, acetic acid, formic acid, trifluoromethanesulfonic acid, methanesulfonic acid, p-toluenesulfonic acid, aluminum chloride, boron trifluoride, zinc chloride, phosphorus pentafluoride, arsenic trifluoride, tin tetrachloride, titanium tetrachloride, antimony pentafluoride and mixtures thereof.

22. The method according to claim 14, wherein the hydrogenation in step b) is carried out in the presence of an acidic cation exchanger.

23. The method according to claim 14, wherein the hydrogenation in step b) is carried out in the presence of an oxidic acidic solid selected from zeolites, silicates, aluminates, aluminosilicates and clays.

24. The method according to claim 14, wherein the catalyst comprises at least one transition metal selected from Pd, Pt, Ni, Rh, Ru, Co, Fe, Zn, Cu, and Re.

25. The method according to claim 14, wherein the hydrogenation catalyst is a supported catalyst.

26. The method according to claim 14, wherein the catalyst support is selected from the group consisting of zirconium dioxide, zinc oxide, magnesium oxide, titanium oxide, aluminum oxide, barium oxide, TiO₂-Al₂O₃, ZrO₂-Al₂O₃, zeolites, hydrotalcite, silicon carbide, tungsten carbide, silicon dioxide, carbon, especially activated carbon or sulfated carbon, diatomaceous earth, clay, barium sulfate, calcium carbonate and mixtures thereof.

27. The method according to claim 14, wherein the temperature in step b) is in the range from 60 to 200° C.

28. The method according to claim 27, wherein the temperature in step b) is in the range from 120 to 150° C.

29. The method according to claim 14, wherein the pressure in step b) is within a range from 900 mbar to 200 bar.

30. The method according to claim 29, wherein the pressure in step b) is within a range from 40 to 80 bar.
